(19) United States
(12) Patent Application Publication
(10) Pub. No.: US 2008/0020029 A1
(43) Pub. Date: Jan. 24, 2008

(54) Drug Delivery System Using an Immune Response System

(75) Inventors: Naoya Kojima, Kanagawa (JP); Yoshitaka Shimizu, Kanagawa (JP); Yuzuru Ikehara, Ibaraki (JP); Hayao Nakanishi, Aichi (JP)

Correspondence Address: GREENBLUM & BERNSTEIN, P.L.C., 1950 ROLAND CLARKE PLACE, RESTON, VA, 20191, US

(73) Assignees: TOKAI UNIVERSITY EDUCATIONAL SYSTEM, Tokyo (JP); AICHI PREFECTURE, Aichi (JP)

(21) Appl. No.: 10/598,916

(22) PCT Filed: Mar. 17, 2005
(86) PCT No.: PCT/JP05/05446

§ 371 Date: Jul. 9, 2007

(30) Foreign Application Priority Data
Mar. 17, 2004 (JP) 2004-076804

(51) Int. Cl. A61K 9/127

(52) U.S. Cl. 424/450

(57) ABSTRACT

The object of the present invention is to provide a drug delivery composition which allows efficient accumulation of an administered substance such as an anti-cancer drug and the like in a target site. The present invention provides a drug delivery liposome composition for delivering a substance to be administered to a target site, which comprises an oligosaccharide coated liposome and a substance to be administered.

### TECHNICAL FIELD

The present invention relates to a drug delivery liposome composition using an oligosaccharide coated liposome. More specifically, the present invention relates to a drug delivery liposome composition using an oligosaccharide coated liposome which is characterized in that it is taken up by a macrophage in the peritoneal cavity and delivered to a target site upon intraperitoneal administration.

### BACKGROUND ART

Post-operative recurrence of cancer is the biggest obstacle that prevents improvement in survival rate of cancer patients, and suppression of the recurrence is one of the most important clinical objects in cancer treatment. The major cause of recurrence after radical operation is considered to be due to free cancer cells that have been already spread at the time of the operation or micrometastasis which cannot be seen macroscopically. Detection and treatment of such micrometastases is an important object which is directly related with the prognosis of cancer patients. For gastric cancer, 50% or more of recurrence cases after radical operations are peritoneal recurrences, which are the most important factor which determines the prognosis of a patient. A positive diagnosis in peritoneal lavage cytodiagnosis, which is the current gold standard, indicates poor prognosis.

However, the sensitivity of detection by the method described above is low because many cases of peritoneal recurrence occurs in many patients with a negative cytodiagnosis, and it is practically impossible to detect peritoneal micrometastasis. Up until now a highly sensitive detection method for free cancer cells in the peritoneal cavity has been established by an RT-PCR method using carcinoembryonic antigen (CEA) as a marker. Further, the results of the analyses using clinical specimens for 8 years since 1995 revealed that a high risk of peritoneal recurrence was directly related to poor prognosis. Currently, with highly advanced medical technology, the risk evaluation for intraperitoneal recurrence and the development of treatment methods for improving the prognosis of gastric cancer patients are under investigation.

A liposome has been used for administration of anti-cancer drugs to improve their therapeutic effect by delivering the anti-cancer drugs to cancer regions more selectively, and also to reduce side effects by suppressing accumulation in normal tissues. A liposome administered in blood vessels has properties of leaking into cancer tissue from the tumor blood vessels which have enhanced vascular permeability and is being retained in a local region. Therefore, the liposome is called a passive targeting system among drug delivery systems. On the other hand, the drug delivery systems using a specific binding activity, such as antibodies, are called active targeting. The objective of conventional methods is to deliver a liposome directly to cancer cells. In such cases, liposomes have been developed so that they are delivered to a cancer region through blood circulation without being taken up by macrophages in blood.

### DISCLOSURE OF THE INVENTION

As described above, detection of peritoneal micrometastasis is in the process of being realized. However, no method is available for specifying the location of a peritoneal micrometastasis. Early intraperitoneal metastasis of gastric cancer is known clinically to start from the greater omentum called milky spots and extranodal small lymph nodes scattered in the mesentery. The present inventors have established a mouse model for micrometastasis, by which micrometastasis occurring in the milky spots can be visualized non-invasively by combining a metastatic cell to which the GFP gene has been introduced and a simple GFP detection system. The present inventors found that the micrometastases were generated in the greater omentum and the lymph nodes of the mesentery, and further found in experiments using mouse that administration of anti-cancer drug in early intraperitoneal metastasis is effective. However, the administration of a drug into a large space of the peritoneal cavity often resulted in a drug concentration not reaching an effective concentration, or if an effective concentration is to be maintained, the drug would have to be administered at a very high concentration, causing secondary problems such as drug transfer in blood, and thus it is not realistic. Therefore, there is no effective administration method at this time. If a drug can be concentrated in a localized area of a peritoneal micrometastasis phase by a drug delivery system, this could be an effective administration method. Thus, the object of the present invention is to provide a drug delivery composition which allows efficient accumulation of an administered substance such as an anti-cancer drug and the like in a target site.

The present inventors investigated vigorously to pursue the object described above, and as a result they found that intraperitoneal administration of an oligomannose coated liposome resulted in very specific and rapid uptake by the resident macrophage in the peritoneal cavity (FIG. 1). Further, it was found that these macrophages which specifically took up the oligomannose coated liposome were accumulated in a short time of 12 to 24 hours in the greater omentun, called the milky spot, and in the extranodal lymph nodes scattered in the mesenteric lymph node, in which early intraperitoneal metastasis was localized (FIG. 2). It was also found that the location where the macrophages, which actually took up the oligomannose coated liposome in the peritoneal cavity, were accumulated was the same as the site where micrometastasis of cancer cells occurred. The present invention has been completed based on these findings.

Thus, the present invention provides a drug delivery liposome composition for delivering a substance to be administered to a target site, which comprises an oligosaccharide coated liposome and a substance to be administered.

Preferably, the oligosaccharide is oligomannose, and more preferably the oligosaccharide is mannopentaose or mannotriose.

Preferably, the substance to be administered is a drug, marker or contrast medium.

Preferably, the drug is an anti-cancer drug.

Preferably, the drug delivery liposome composition of the present invention is administered intraperitoneally, taken up by macrophage in the peritoneal cavity and delivered to a target site.

Preferably, the target site is the extranodal small lymphatic tissue in the peritoneal cavity or the lymphatic tissue in the mesentery.

Preferably, the drug delivery liposome composition of the present invention is administered in combination with oligosaccharide coated liposome encapsulating a magnetic compound.

Another aspect of the present invention provides a method for delivering a substance to be administered to a target site, which comprises administering a drug delivery liposome composition comprising an oligosaccharide coated liposome and the substance to be administered to mammals including humans.

Preferably, the drug delivery liposome composition of the present invention is administered to mammals including humans, in combination with an oligosaccharide coated liposome encapsulating a magnetic compound, and then a magnetic field can be applied externally.

### BRIEF DESCRIPTION OF THE DRAWINGS

FIG. 1 shows the results of observation of the uptake of a liposome coated with M3-DPPE and a liposome not coated with M3-DPPE by the cells in the peritoneal cavity (F4/80 positive cells). The liposome coated with M3-DPPE and the liposome not coated with M3-DPPE were administered to mice, and cells were collected 1 hour later and observed;

FIG. 2 shows the result of time-course observation of the accumulation of the M3-DPPE coated liposome in the greater omentum;

FIG. 3 shows the result of the investigation of the optimum uptake conditions into the greater omentum for oligosaccharide coated liposome encapsulating an anti-cancer drug and oligosaccharide coated liposome encapsulating magnetized fine particles;

FIG. 4 shows the result of observation of the growth of cancer using fluorescence of GFP as an index in mice receiving or not receiving an anti-cancer drug (5FU) and then subjected to celiotomy;

FIG. 5 shows the result of observation of the growth of cancer using fluorescence of GFP as an index in mice receiving or not receiving an anti-cancer drug (5FU) and then subjected to celiotomy; and

FIG. 6 shows the result of observation of the growth of cancer using fluorescence of GFP as an index in mice receiving or not receiving an anti-cancer drug (5FU) and then subjected to celiotomy.

### BEST MODE FOR CARRYING OUT THE INVENTION

The mode for carrying out the present invention will be described specifically below.

The drug delivery liposome composition of the present invention is characterized by comprising an oligosaccharide coated liposome and a substance to be administered and is used for delivering the substance to be administered to a target site. More particularly, the drug delivery liposome composition of the present invention is taken up by macrophages in the peritoneal cavity when administered into the peritoneal cavity, and is then delivered to the target site. The preferred target site of the present invention is the greater omentum and the extranodal small lymphatic tissue in the mesentery, which are the early intraperitoneal metastasis lesions for cancer.

The oligosaccharide coated liposome to be used in the present invention includes, for example, liposomes described in JP Patent No.2828391. The types of the sugar components which constitute the oligosaccharide are not limited and include, for example, D-mannose (D-Man), L-fucose (L-Fuc), D-acetylglucosamine (D-GlcNAc), D-glucose (D-Glc), D-galactose (D-Gal), D-acetylgalactosamine (D-GalNAc), D-rhamnose (D-Rha) and the like.

In the oligosaccharide, each sugar component is linked by α1→2, α1→3, α1→4, α1→6, α1→4 linkage or the like, or by a combination thereof. For example, mannose may form a single chain by the linkage described above, or a branched chain by a combination of α1→3 and α1→6 linkages. The preferred number of monosaccharide in oligosaccharide is 2 to 11. Specific examples of oligosaccharides include mannobiose (Man2), mannotriose (Man3), mannotetraose (Man4), mannopentaose (Man5), mannohexaose (Man6), mannoheptaose (Man7), and various mixed oligosaccharides, for example, M5 (Formula 1) and RN (Formula 2) described below and the like. 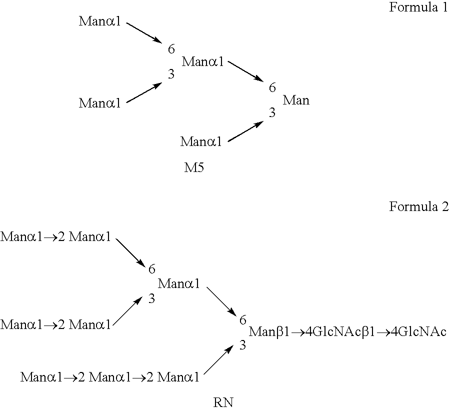
(wherein, α1→2 linked Man's may be, independently, present or not present.)

Further, the oligosaccharide containing glucose may include the substance having the structure represented by Formula 3. The oligosaccharide containing N-acetylglucosamine may include the substance having the structure represented by Formula 4. The oligosaccharide containing fucose may include the substance having the structure represented by Formula 5. 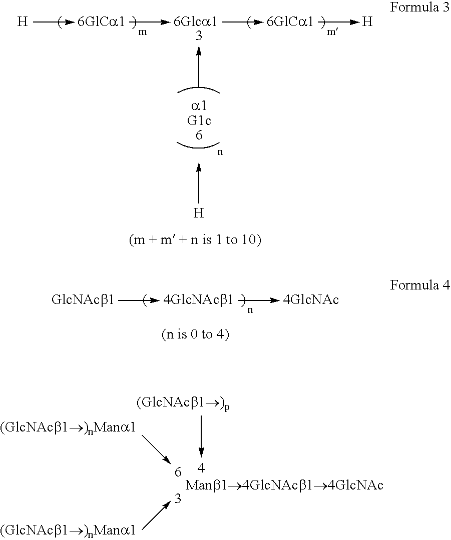
(p is 0 or 1 and each n is independently 0 to 3. Each of the 2 GlcNAc residues represented by 4GlcNAcβ1→4GlcNAc in the right side of the formula may be independently present or not present. Further, every GlcNAc represented by (GlcNAcβ1→)ₙ may be linked to any of the free hydroxyl group on the adjoining mannose on the right side through glycoside linkage.)

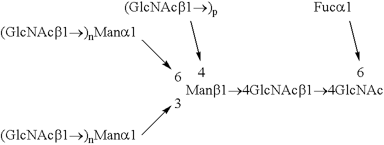

(p is 0 or 1 and each n is independently 0 to 3. Further, every GlcNAc represented by (GlcNAcβ1→)ₙ may be linked to any of the free hydroxyl group on the adjoining mannose on the right side through glycoside linkage.)

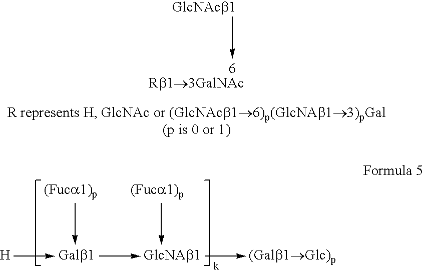

(k is 1-5 and each p is independently 0 or 1. The arrows without a number on the arrow head may be linked to any of the free hydroxyl group through glycoside linkage.)

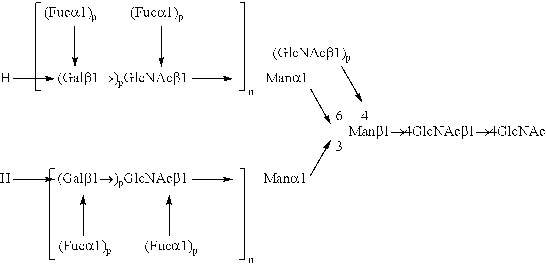

(Each p is independently 0 or 1, and each n is independently 0 to 3. The arrows without a number on the arrow head may be linked to any of the free hydroxyl group through glycoside linkage. Also, each of the 2 GlcNAc residues represented by 4GlcNAcβ1→4GlcNAc in the right side of the formula may be independently present or not present.)

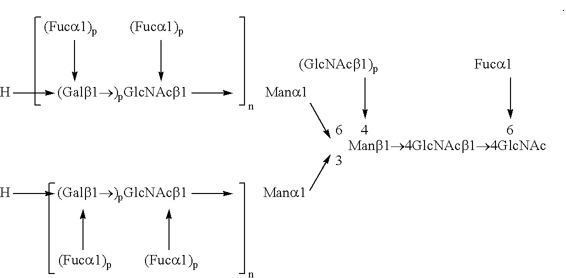

(Each p is independently 0 or 1, and each n is independently 0 to 3. The arrows without a number on the arrow head may be linked to any of the free hydroxyl group through glycoside linkage. Each of the 2 GlcNAc residues represented by 4GIcNAcβ1→4GlcNAc in the right side of the formula may be independently present or not present.)

The oligosaccharide used in the present invention is preferably oligomannose, and mannopentaose or mannotriose is especially preferable.

Any of the oligosaccharides described above contains one reducing terminal aldehyde group. Thus this aldehyde group can be utilized as a means for introducing the oligosaccharide to the surface of a liposome. That is, a Schiff base is formed by reacting this aldehyde with a lipid containing amino groups, and then the oligosaccharide and the lipid can be linked by reducing the Schiff base with a standard method, preferably by chemical reduction, for example, by NaBH₃CN (Mizuochi, Tsuguo, Carbohydrate Engineering, pp 224-232, Industrial Research Center, Biotechnology Information Center, 1992).

The phospholipid containing amino groups, for example, phosphatidylamine such as dipalmitoylphosphatidyl-ethanolamine (DPPE), distearoylphosphatidyl-ethanolamine (DSPE) and the like may be used preferably as the lipid containing amino groups described above. The linked substance of oligosaccharide and lipid obtained as described above may be referred to as an artificial glycolipid in the present invention.

Any known lipid conventionally known to be used to constitute liposome can be used singly or in combination for constituting the liposome. For example, natural products such as lipids obtained from egg yolk, soy bean or any other animals and plants, modified products of these lipids, such as hydrogenation products with a decreased degree of unsaturation or chemically synthesized products may be used. In particular, the examples include: sterols such as cholesterol (Chol); phosphatidyl-ethanolamines such as dipalmitoylphosphatidyl-ethanolamine (DPPE) and distearoylphosphatidyl-ethanolamine (DSPE); phosphatidylcholines such as dipalmitoylphosphatidyl-choline (DPPC) and distearoylphosphatidyl-choline (DSPC); phosphatidyl serines such as dipalmitoylphosphatidyl-serine (DPPS) and distearoylphosphatidyl-serine (DSPS); phosphatidic acids such as dipalmitoyl phosphatidic acid (DPPA) and distearoyl phosphatidic acid (DSPA); and the like.

Liposomes can be prepared using a known method [D. W. Deeamer, P. S. Uster, “Liposome” ed. by M. J. Ostro, Marcel Dekker Inc., N.Y. Basel, 1983, p27]. In general the vortex method and ultrasonic method are used, but other methods such as the ethanol injection method, ether method and reverse phase evaporation method may be applied, and these methods may be used in combination.

For example, in the vortex method and the ultrasonic method, a predetermined lipid is dissolved in an organic solvent, such as methanol, ethanol, chloroform or a mixture thereof, for example a mixture of methanol and chloroform, and then a thin layer of the lipid is obtained by evaporating the organic solvent off. Subsequently, an aqueous medium is added to the lipid thin layer, and the liposome is formed by vortex or ultrasonic treatment. During this process, a substance to be administered can be encapsulated in the liposome by mixing the substance to be administered such as a drug, marker or contrast medium with the aqueous medium, for example by dissolving or suspending.

Introduction of an oligosaccharide to the surface of the liposome may be carried out by choosing, for example, any one of the following two methods. If the aforementioned artificial glycolipid is water soluble and not dissolved in organic solvent sufficiently, and if, for example, the aforementioned linked product between M5 and DPPE (M5-DPPE) or RN and DPPE (RN-DPPE), are used, the aqueous solution of these products may be prepared, and mixed with the liposome formed, and the mixture is incubated for example at 4° C. or at room temperature for 24-120 hours, for example 72 hours.

On the other hand, if the artificial glycolipid is soluble in organic solvents, this artificial glycolipid may be dissolved in the aforementioned organic solvent together with the lipid which constitutes the liposome during the liposome production process, and subsequently the liposome may be formed according to the standard method. The amount of oligosaccharide to be added to the liposome may vary depending on the kind of oligosaccharide, the kind of antigen to be encapsulated, the structure of the combination of liposome and the like, but in general, it is 5 μg-500 μg for 1 mg of lipid which constitutes the liposome.

The liposome used in the present invention may be a multilayer type (multilamella vesicle) or a monolayer type (unilamella vesicle). These can be prepared according to the known standard method. Further, one type can be converted to the other type according to the standard method. For example, the multilamella vesicle type liposome can be converted to the unilamella vesicle type liposome. The particle diameter of the liposome used in the present invention is not particularly limited, and the particle size can be adjusted by the standard method as needed, for example, by filtering through a filter having the desired pore size.

The substance to be administered to be used in the present invention is preferably a drug, marker or contrast medium. Examples of drug include anti-cancer drug, cancer vaccine, antigen peptide, immuno-activating agent (for example, Picibanil and the like), cytokine and inhibitor of angiogenesis.

The kind of anti-cancer drug which can be used in the present invention is not particularly limited and includes: alkylating drug (for example, cyclophosphamide, nimustine hydrochloride, ifosfamide, ranimustine, thiotepa, melphalan, busulfan, dacarbazine, carboquone, procarbazine hydrochloride and the like); antimetabolites (for example, cytarabine, tegafur, cytarabine ocfosfate, enocitabine, fludarabine phosphate, levofolinate calcium, gemcitabine hydrochloride, methotrexate, mercaptopurine, carmofur, 6-mercaptopurine riboside, hydroxycarbamide, fluorouracil, folinate calcium, doxifluridine and the like); molecular target drugs (tyrosine kinase inhibitor); or alkaloids (vincristine sulfate, vindesine sulfate, vinblastine sulfate and the like).

Examples of marker include fluorescent proteins such as GFP and fluoro deoxy glucose. Further, examples of the contrast media include non-ionic aqueous iodine, aqueous iodine and low osmotic pressure aqueous iodine contrast medium.

The amount of the substance to be administered for the amount of a liposome is not particularly limited as long as the effect of the present invention is obtained so that the liposome composition administered is taken up by the macrophage in the peritoneal cavity and delivered to the target site, and it may be set appropriately according to the kind of the substance to be administered, the composition and structure of the liposome. In general the amount of the substance to be administered is 1 [μg-100 μg per 1 mg of lipid which constitutes the liposome.

The liposome composition of the present invention may comprise a pharmaceutically acceptable carrier as desired. Sterile water, buffer solution or saline may be used as the carrier. Also, the liposome composition of the present invention may comprise salts, saccharides, protein, starch, gelatin, vegetable oil, polyethylene glycol and the like as desired.

The administration route of the liposome composition of the present invention is not particularly limited, but it can be preferably administered intraperitoneally. The amount of administration of the liposome composition of the present invention varies depending on the kind of a substance to be administered, administration route, severity of symptoms, age and conditions of a patient, degree of side effects and the like, but in general it is in the range of 0.1-100 mg/kg/day.

The liposome composition of the present invention can be administered together and in combination with the oligosaccharide coated liposome encapsulating a magnetic compound. The magnetic compound to be used in the present invention is preferably a magnetic fine particle which generates heat or oscillates under a magnetic field. In this case, a mixture obtained by mixing the liposome composition containing the oligosaccharide coated liposome and an anti-cancer drug, and the liposome containing the oligosaccharide coated liposome and the magnetic compound can be administered to a living body. In this case, the anti-cancer drug can be released from the macrophages which phagocytosed these liposome compositions incorporated in the greater omentum by applying an external magnetic field, and thus it becomes possible to suppress effectively the tumor tissue which metastasized to this site.

Next, a method for utilizing the drug delivery liposome composition of the present invention will be described.

(1) Drug Delivery System for Anti-cancer Drug to the Extranodal Lymphatic Tissue in the Peritoneal Cavity using Peritoneal Macrophages as a Carrier

When M3 liposome (FITC-BSA is encapsulated) is administered intraperitoneally, it accumulates in the greater omentum and the lymphatic tissues in the mesentery (milky spots) with the passage of time. In mice in which the peritoneal immune system is disrupted, a portion of the liposome is delivered to the spleen, but otherwise almost no incorporation to macrophages in the spleen is observed. Therefore, by encapsulating an anti-cancer drug to this liposome, it becomes possible to accumulate the anti-cancer drug and to act on the early metastatic lesion in the peritoneal lymph node. Effective anti-cancer drugs often show strong side effects, and various drug delivery systems have been devised to improve this point. Since the anti-tumor effect is, in general, dependent on a drug concentration in a tumor, the technique of accumulating an anti-cancer drug in a tumor site by using an M3 liposome can be utilized widely as the delivery system for anti-cancer drugs. The system of the present invention is based on the following 3 steps of the immunological mechanism.

(i) An M3 liposome containing mannose conjugated on the surface is specifically and quickly phagocytosed by encountering macrophages, and is accumulated in the lysosome.

(ii) Intracellular uptake through the mannose receptor activates the macrophages. Due to this activation, macrophages accumulate at the marginal sinus of the regional lymph node for antigen presentation.

(iii) The macrophages which reach the lymph node secrete a substance which cannot be digested in the lysosome to outside of the adhesion surface of the cell.

By using this method, high concentrations of the anti-cancer drug accumulate efficiently at the tumor site. After accumulation, the anti-cancer drug is slowly secreted from the macrophages over a long period of time and only the tumor site can be thus exposed to the anti-cancer drug over a long period of time. Further, by giving controlled stress such as heat and the like extracorporeally to the accumulated macrophages, the anti-cancer drug can be secreted vigorously and actively.

(2) Cancer Vaccine Delivery System to the Extranodal Lymphatic Tissue in the Peritoneal Cavity using Peritoneal Macrophages as Carrier

The use of the oligomannose coated liposome is a technique which can be applied for a cancer vaccine. It is believed that the efficacy of the cancer vaccine is dependent on how to input the information of tumor antigen efficiently to antigen presenting cells so that the immune activity which attack cancer cells is induced more effectively. With regard to this point, when the cancer antigen and adjuvant are encapsulated in the oligomannose coated liposome and are sprayed in the peritoneal cavity, these drugs are delivered by macrophages to reach the regional lymphatic tissues which are the metastatic focus of cancer and can stimulate local immune activity. The low efficacy of vaccine due to insufficient activation of immune reaction, which has been a problem in immune therapy for cancer until now, can be improved by activating anti-tumor immunity in the local site in a cancer lesion.
(3) Detection of a site with a risk of intraperitoneal early metastasis by the oligomannose coated liposome encapsulating a fluorescent substance and the like

Even if the presence of intraperitoneal free cancer cells are confirmed by the detection method with high sensitivity using RT-PCR and the high probability of peritoneal micrometastasis is suspected, the survival rate is only about 50%. This is not unrelated to the fact that the location of the peritoneal micrometastasis cannot be specified. Because the site where macrophages, in which the oligomannose coated liposome is taken up, are accumulated and the site where micrometastasis of cancer cells occurs are the same, it is possible to detect the site where the peritoneal micrometastasis occurs with a high frequency by administrating a liposome, which encapsulates a substance which is easily recognizable during the operation, such as fluorescent protein and the like, 24 hours before the operation. This makes it possible to resect with minimum invasion prophylactically.

(4) Other Applications

(A) Application to the Treatment for Lymph Node Metastasis of Cancer

In breast cancer, which is increasing in number in recent years, lymph node metastasis greatly affects the prognosis of a patient. Because the prognosis is not improved even with wide resection of the lymph nodes, the main treatment methods are shifting to a combination of reduction surgery and chemotherapy. Since the axillary, supraclavicular, and parastemal lymph nodes are the regional lymph nodes for breast cancer, recurrence from these lymph nodes is occasionally observed. By injecting an M3 liposome containing an anti-cancer drug or, as cancer immunotherapy, an M3 liposome containing a cancer antigen and an adjuvant near the lesion after the operation, effective drug delivery to the regional lymph node by macrophages is expected, and a good effect for the drug therapy is further expected. Apart from this, based on a similar mechanism, this treatment method can be applied to melanoma, thyroid cancer and lung cancer which are prone to lymph node metastasis.

(B) Application to Hematologic Tumors

In hematologic tumors, the targets for treatment are tumors showing monocyte and macrophage differentiation. If the anti-cancer agent encapsulated in the M3 liposome of the present invention has a good molecular targeting characteristic, even if it is incorporated into a macrophage other than the tumor, side effects can be reduced, and a drug effect limited to the tumor cells can be anticipated.

The present invention will be described more concretely with the following examples. The present invention is not limited to these examples.

### EXAMPLES

### Example 1

### : A Method for Production of an Oligosaccharide Coated Liposome and a Method for Encapsulating a Drug, Marker or Contrast Medium

Mannopentaose (M5) (the compound represented by Formula 1) or mannotriose (M3) (Mannotriose (Man3) represented by the structure Manα1→6 (Manα1→3) Man) and dipalmitoylphosphatidyl-ethanolamine (DPPE) were linked by the reductive amination reaction to synthesize M5-DPPE and M3-DPPE according to the method below.

First, to prepare an oligosaccharide solution, 2.5 mg of mannopentaose (M5) or mannotriose (M3) was mixed with 600 μl of distilled water and the mixture was stirred to dissolve. Next, to prepare the DPPE solution, DPPE was dissolved in a mixture of chloroform/methanol (1:1 by volume) at a concentration of 5 mg/ml. Also, NaBH₃CN was dissolved in methanol at a concentration of 10 mg/ml to prepare an NaBH₃CN solution. To 600 μl of each of the aforementioned oligosaccharide solutions, 9.4 ml of the aforementioned DPPE solution and 1 ml of the aforementioned NaBH₃CN solution were added and mixed with stirring. This reaction mixture was incubated at 60° C. for 16 hours to generate an artificial glycolipid. The artificial glycolipid thus prepared was purified to high purity using HPLC.

A liposome encapsulating TRITC labeled protein (Example 2), or FITC or rhodamine labeled protein (Example 3) was prepared as follows.

First, dipalmitoylphosphatidyl-choline (DPPC), cholesterol and artificial glycolipid (M5-DPPE or M3-DPPE) were mixed at 1:1:0.1 in a chloroform/methanol or ethanol solution and the mixture was poured into a pear shaped flask and evaporated to dryness under reduced pressure by a rotary evaporator to prepare lipid film. Next, 0.3 ml of a PBS solution containing TRITC labeled protein (Example 2), or FITC or rhodamine labeled protein (Example 3) at 5 mg/ml was added to the lipid film, and the mixture was stirred vigorously using a vortex mixer to prepare an M5-DPPE coated liposome or M3-DPPE coated liposome. FITC-BSA or TRITC-BSA was used as the TRITC labeled protein, or the FITC or rhodamine-labeled protein.

Subsequently, liposome was washed several times with PBS, and soluble substances not encapsulated in liposome were removed by centrifugation. Further, the particle size of the liposome was adjusted by using a 1 μm filter. The mass of encapsulated protein was measured by a protein assay method, and the lipids composition ratio of the liposome and the drug were assayed by HPLC.

### Example 2

### An Evaluation Method for Macrophage Incorporation and a Brief Description of the Results

An M5-DPPE coated liposome or M3-DPPE coated liposome, in which TRITC labeled BSA was encapsulated, was administered intraperitoneally to mice (100 microgram as cholesterol), and the peritoneal cells were recovered by the standard method 30, 60, 120 and 180 minutes later. Recovered cells were stained with FITC labeled anti CD11c antibody or F4/80, and then the fluorescence intensity of rhodamine incorporated into the cells and cell surface antigen (FITC) was analyzed using FACS.

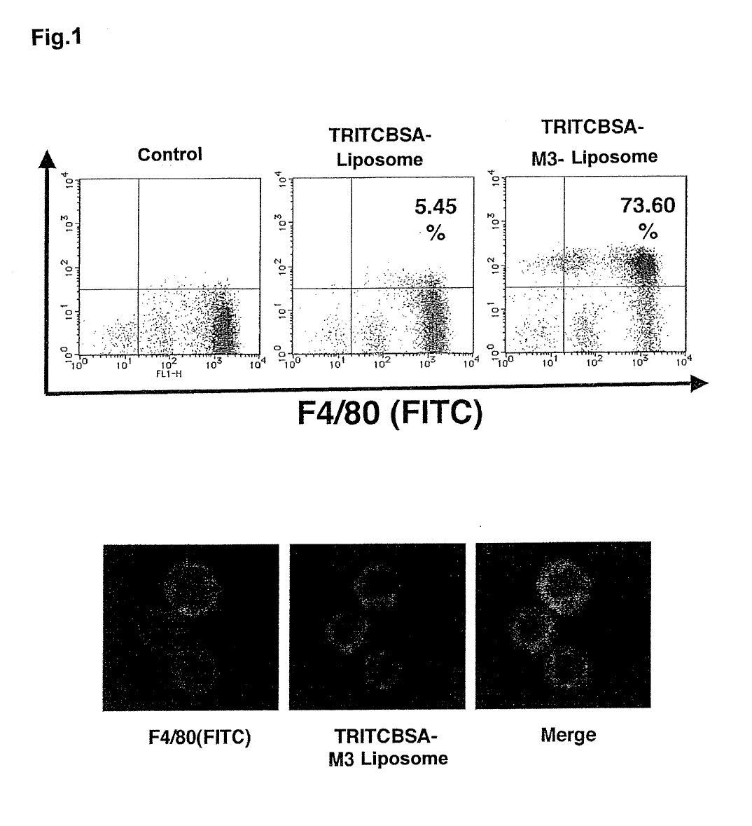

FIG. 1 shows the view of the incorporation into the peritoneal cells which were recovered 1 hour after administration of M3-DPPE coated liposome and M3-DPPE uncoated liposome. When the M3-DPPE coated liposome was administered, 78% of the cells stained by the macrophage marker, F4/80, had strong fluorescence of TRITC, indicating that the M3-DPPE coated liposome encapsulating TRITC labeled protein was taken up by macrophages. On the other hand, almost no uptake was observed when the liposome not coated with M3-DPPE was administered. As shown in FIG. 1, lower figure, the M3-DPPE coated liposome is taken up by macrophages as granules.

### Example 3

### An Evaluation Method for Accumulation of Macrophage or Liposome on the Target Site and a Brief Description

100 micrograms (converted to cholesterol) of the M3-DPPE coated liposome encapsulating FITC or rhodamine labeled protein was diluted with physiological saline, and a total volume of 0.5 ml was inoculated intraperitoneally to nude mice. Subsequently mice were sacrificed at various time points (3, 6, 12 and 24 hours later) and observed. After performing celiotomy on mice, the upper abdomen, which included the greater omentum in the peritoneal cavity of the mice, was irradiated with blue light (150 W halogen light source, LGPS-2, equipped with a 420-480 band pass filter). The image of the accumulation of the M3 liposome in the greater omentum by a stereoscopic microscope (Olympus GFP Specific Checker, SZ40-GFP) equipped with a yellow filter (a long pass filter which passes visible light of 500 nm or a longer wavelength) under the dark field was outputted through a digital camera as green color (FITC) to a personal computer and evaluated.

Samples with rhodamine were observed using a 150 W halogen light source, a band-pass filter 545-580 and a long pass filter (590 nm or above) as an absorption filter.

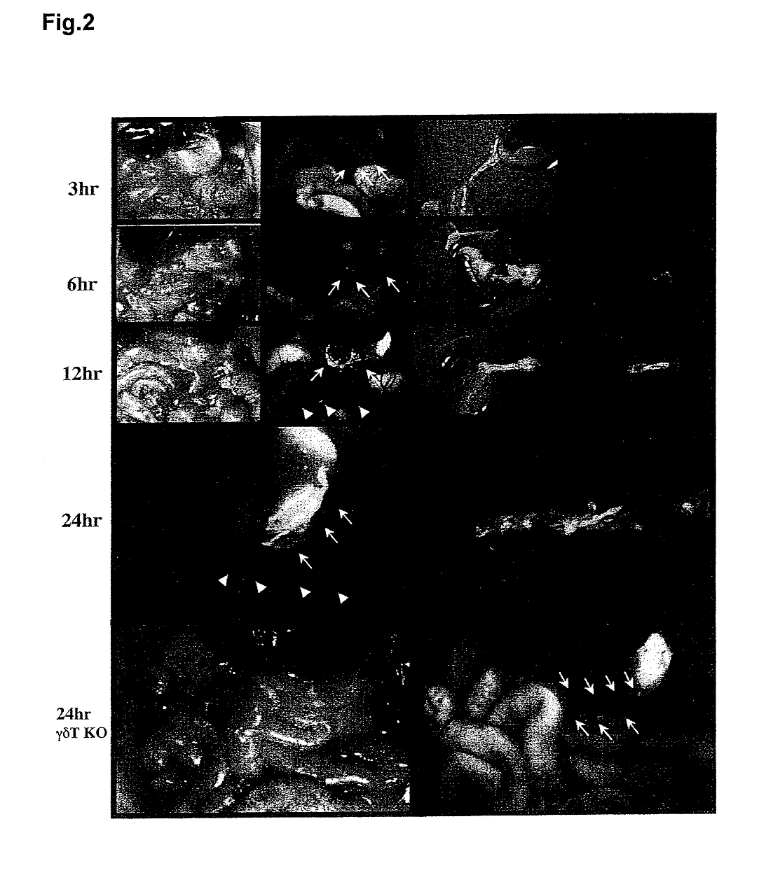

FIG. 2 shows the time-course accumulation of the M3-DPPE coated liposome in the greater omentum. The accumulation was already observed 3 hours later, reached a maximum 12 hours later and observed up until 24 hours thereafter. Since very little accumulation was observed in γδT cell deletion mice in which the extranodal lymphatic tissue is poorly formed, it is formed that the M3-DPPE coated liposome is accumulated in the extranodal lymphatic tissue. On the other hand, accumulation of the liposome not coated with M3-DPPE was hardly seen.

### Example 4

### Experiments Confirming Anti-Cancer Effect on Peritoneal Metastasis of Gastric Cancer with the Liposome Encapsulating an Anti-Cancer Drug and the Liposome Encapsulating Magnetic Fine Particles

(1) Liposome accumulation in the greater omentum by administering a mixture of the oligosaccharide coated liposome encapsulating an anti-cancer drug and the oligosaccharide coated liposome encapsulating magnetic fine particles

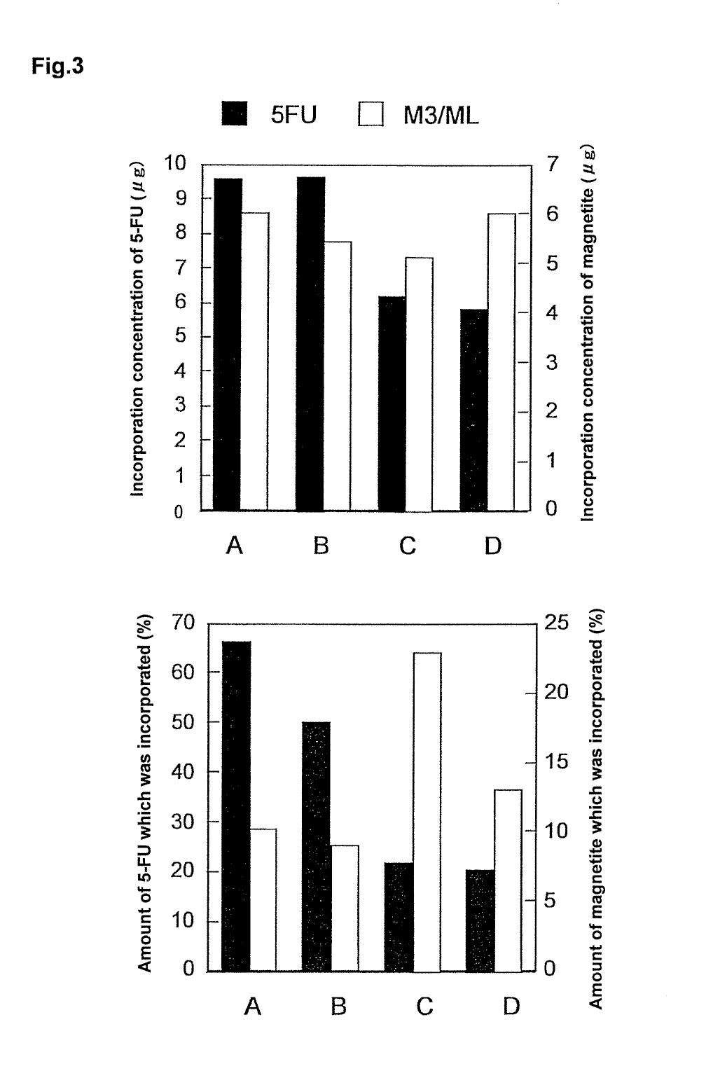

The oligosaccharide coated liposome encapsulating an anti-cancer drug (120 μg/ml of 5FU, 2 mg/ml of cholesterol) and the oligosaccharide coated liposome encapsulating magnetic fine particles (1.5 mg/ml of magnetite, 2 mg/ml of cholesterol) were prepared, mixed at a ratio shown below and administered to mice intraperitoneally. At 24 hours later the greater omentum was excised from the mice, and 5FU and iron ions therein were measured (FIG. 3).
A: M3/5-FU containing 240 μg of Cholesterol

M3/ML containing 20 μg of cholesterol
B: M3/5-FU containing 320 μg of cholesterol

M3/ML containing 40 μg of cholesterol
C: M3/5-FU containing 480 μg of cholesterol

M3/ML containing 20 μg of cholesterol
D: M3/5-FU containing 480 μg of cholesterol

M3/ML containing 40 μg of cholesterol
5-FU concentration: 120 μg/ml; M3/ML concentration: 1.5 mg/ml; cholesterol: 2 mg/ml

The results indicated that administration of a mixture of the oligosaccharide coated liposome encapsulating anti-cancer drug at 240 μg of cholesterol and the oligosaccharide coated liposome encapsulating magnetic fine particles at 20 μg of cholesterol gave the best accumulation efficacy.

(2) After investigating the administration condition described above, the anti-cancer effect was investigated.

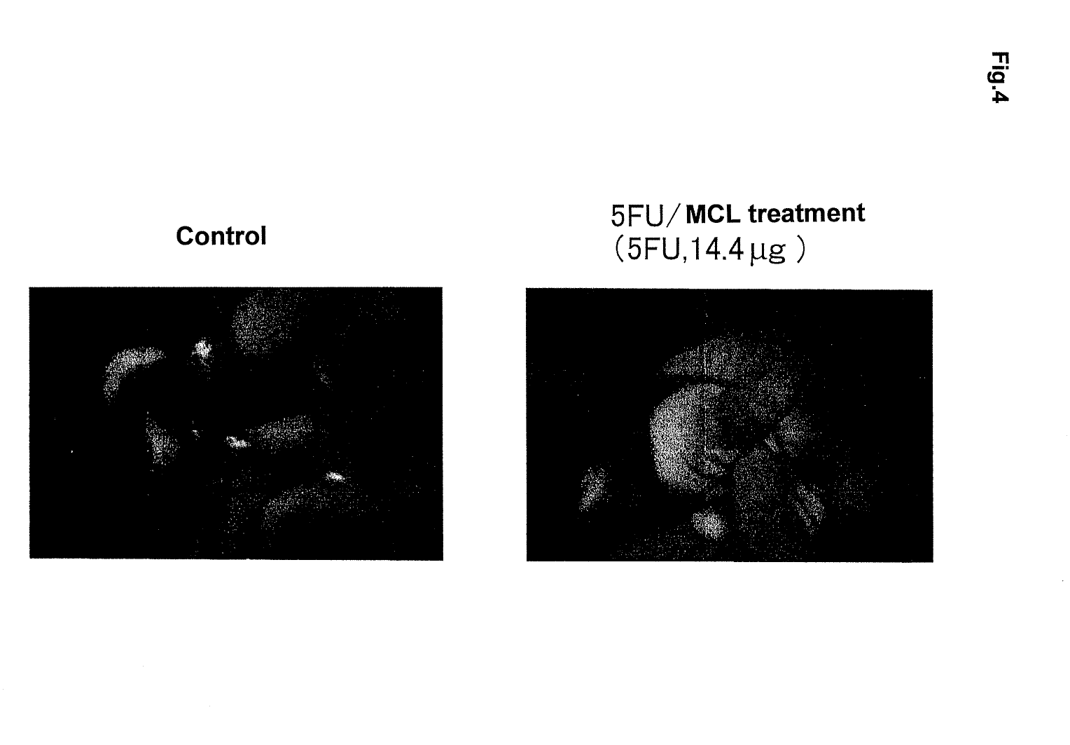

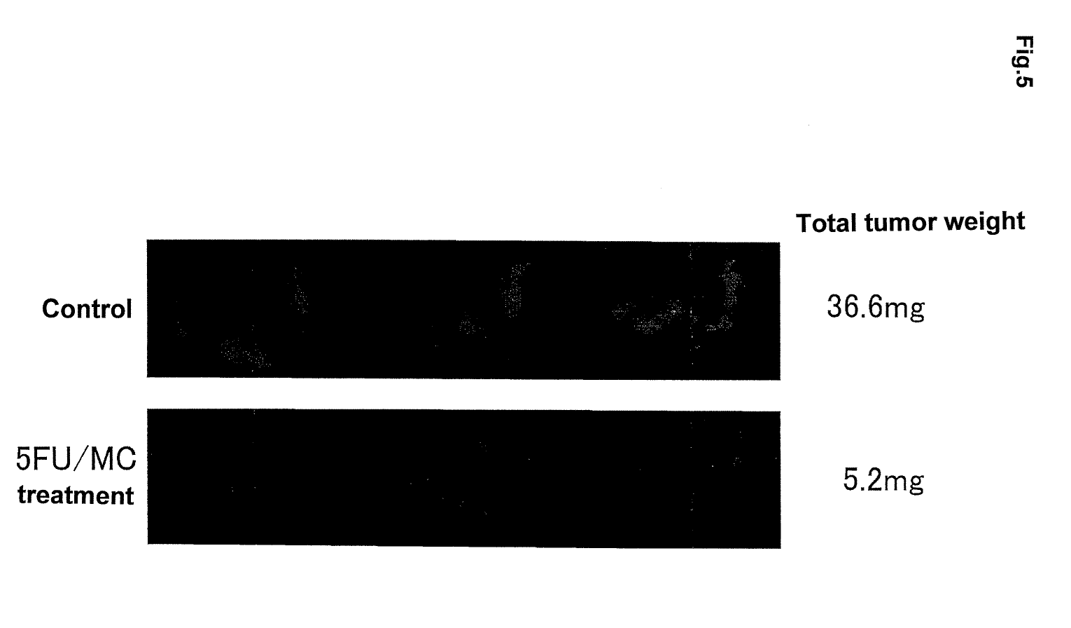

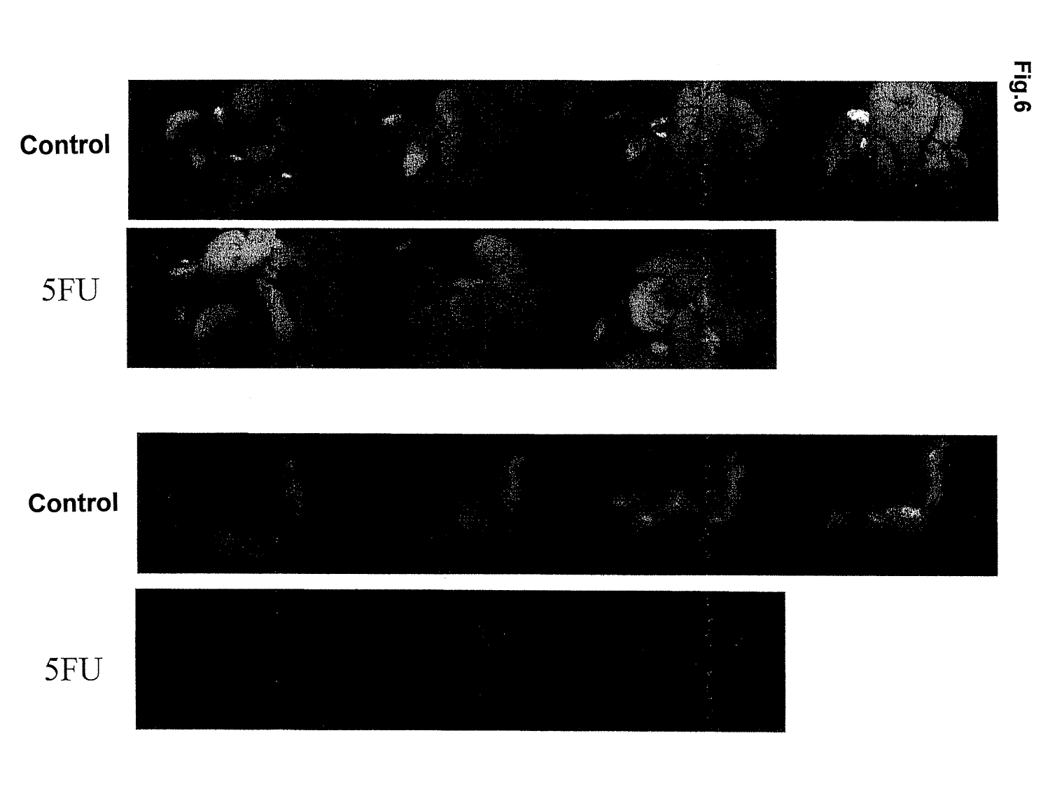

First, 3×10⁶ cells of the gastric cancer cell strain MKN28, in which GFP was introduced, were administered intraperitoneally to nude mice. At 24 hours later, engraftment of the cancer cells was confirmed using the fluorescence of GFP as a marker. The oligosaccharide coated liposome encapsulating anti-cancer drug at 240 μg of cholesterol and the oligosaccharide coated liposome encapsulating magnetic fine particles at 20 μg of cholesterol were mixed and administered intraperitoneally to mice in which engraftment was confirmed. At 24 hours after the liposome administration, alternating magnetic field irradiation was carried out for 30 minutes using an Alternating Magnetic Field Irradiation Apparatus (Dai-Ichi High Frequency Co., Ltd.) and a High Frequency Induction Heating (Fuji Electronic Industrial Co. Type F1H-153HH, Output: 15 Kw, 400 KHz). One week later, the mice were subjected to celiotomy, and the growth of the cancer was checked using the GFP fluorescence as a marker, and the weight of the tumor was measured. The method described above and the results are shown in FIG. 4 to FIG. 6. The tumor weight was 36.6 mg in the control mice and 5.2 mg in the mice treated with the anti-cancer drug (5FU), demonstrating that the tumor weight was markedly reduced by the administration of the liposome composition of the present invention. GFP fluorescence observation also indicated that the growth of the cancer was suppressed in the mice treated with the anti-cancer drug (5FU).

### Industrial Applicability

The present invention can provide a drug delivery liposome composition which can efficiently accumulate and release substances to be administered such as an anti-cancer agent and the like to a target site.

1. A drug delivery liposome composition for delivering a substance to be administered to a target site, which comprises an oligosaccharide coated liposome and a substance to be administered.

2. The drug delivery liposome composition of claim 1 wherein the oligosaccharide is oligomannose.

3. The drug delivery liposome composition of claim 1 wherein the oligosaccharide is mannopentaose or mannotriose.

4. The drug delivery liposome composition of claim 1 wherein the substance to be administered is a drug, marker or contrast medium.

5. The drug delivery liposome composition of claim 4 wherein the drug is an anti-cancer drug.

6. The drug delivery liposome composition of claim 1 which is administered intraperitoneally, taken up by macrophage in the peritoneal cavity and delivered to a target site.

7. The drug delivery liposome composition of claim 1 wherein the target site is the greater omentum and the extranodal small lymphatic tissue in the mesentery, which are the early intraperitoneal metastasis lesions for cancer.

8. The drug delivery liposome composition of claim 1 which is administered in combination with oligosaccharide coated liposome encapsulating a magnetic compound.
